(19) United States
(12) Patent Application Publication
(10) Pub. No.: US 2020/0008751 A1
(43) Pub. Date: Jan. 9, 2020

(54) MEDICAL INFORMATION PROCESSING APPARATUS

(71) Applicant: Canon Medical Systems Corporation, Otawara-shi (JP)

(72) Inventors: Yusuke KANO, Nasushiobara (JP); Anri Sato, Nasushiobara (JP)

(73) Assignee: Canon Medical Systems Corporation, Otawara-shi (JP)

(21) Appl. No.: 16/446,819

(22) Filed: Jun. 20, 2019

(30) Foreign Application Priority Data
Jul. 4, 2018 (JP) 2018-127879
Jun. 11, 2019 (JP) 2019-108388

(51) Int. Cl. A61B 5/00; G16H 10/60; G16H 50/20

(52) U.S. Cl. CPC A61B 5/7275; G16H 10/60; G06N 5/025; A61B 5/746; G16H 50/20

(57) ABSTRACT

A medical information processing apparatus according to an embodiment includes processing circuitry. The processing circuitry is configured to obtain, from among medical data related to subjects, medical data in a specific time period at or earlier than a previous point in time that is earlier by a predetermined length of time than a time of occurrence of a predetermined state change in the subjects. The processing circuitry is further configured to generate, on the basis of information about the medical data in the specific time period, learning-purpose data used for generating a learned model configured to output information about the predetermined state change in a subject being subject to a prediction that may occur at a time later by the predetermined length of time than a time at which a medical service is provided for the subject.

### CROSS-REFERENCE TO RELATED APPLICATIONS

This application is based upon and claims the benefit of priority from Japanese Patent Application No. 2018-127879, filed on Jul. 4, 2018; and Japanese Patent Application No. 2019-108388, filed on Jun. 11, 2019, the entire contents of all of which are incorporated herein by reference.

### FIELD

Embodiments described herein relate generally to a medical information processing apparatus.

BACKGROUND

At medical sites, conventionally, patients have a possibility of abruptly experiencing a sudden change in the clinical state such as heart failure. For example, when patients are infants having a congenital heart disease, it is not unusual to abruptly have an unexpected sudden change in the clinical state. It may be possible in some situations to recognize an advance indication of such a sudden change in the clinical state by looking at clinical states or medical records retrospectively; however, there are many situations where such an advance indication goes unnoticed during an actual diagnosing process.

One of the causes may be, for example, that it is difficult to visually recognize changes when observing raw data because pediatric medical data has various levels of reference values and different baselines depending on how old (months/years) the children are. Further, for example, because sensors are easily affected by body movements and crying, pediatric medical data have many outlier values in the data, which leads to another problem where there would be frequent erroneous detection if deterioration in the clinical state was detected by simply using a threshold value or the like.

### BRIEF DESCRIPTION OF THE DRAWINGS

FIG. 1 is a diagram illustrating an exemplary configuration of a medical information processing apparatus according to present embodiments;

FIG. 2 is a chart illustrating processes performed in a learning mode and an operation mode by processing circuitry according to a first embodiment;

FIG. 3 is a table of National Early Warning Scores (NEWS) used as an example of medical data according to the first embodiment;

FIG. 4 presents charts illustrating examples of control chart abnormality judgement rules used as another example of the medical data according to the first embodiment;

FIG. 5 is a chart illustrating a first example related to processes in the learning mode performed by the processing circuitry according to the first embodiment;

FIG. 6 is a chart illustrating a second example related the processes in the learning mode performed by the processing circuitry according to the first embodiment;

FIG. 7 is a chart illustrating a third example related to tie processes in the learning mode performed by the processing circuitry according to the first embodiment;

FIG. 8 is a drawing illustrating an example of a screen displayed on a display by a controlling function according to the first embodiment;

FIG. 9 is a flowchart illustrating a processing procedure of processes in the learning mode performed by the medical information processing apparatus according to the first embodiment;

FIG. 10 is a flowchart illustrating a processing procedure of processes in the operation mode performed by the medical information processing apparatus according to the first embodiment;

FIG. 11 is a chart illustrating processes in a learning mode and an operation mode performed by processing circuitry according to a second embodiment;

FIG. 12 is a chart illustrating an example related to processes in the learning mode performed by the processing circuitry according to the second embodiment;

FIG. 13 a chart illustrating another example related to the processes in the learning mode performed by the processing circuitry according to the second embodiment;

FIG. 14 is a flowchart illustrating a processing procedure of processes in the learning mode performed by a medical information processing apparatus according to the second embodiment; and

FIG. 15 is a flowchart illustrating a processing procedure of processes in the operation mode performed by the medical information processing apparatus according to the second embodiment.

### DETAILED DESCRIPTION

A medical information processing apparatus according to an embodiment includes an obtaining unit and a generating unit. The obtaining unit is configured to obtain, from among medical data related to subjects, medical data in a specific time period at or earlier than a previous point in time that is earlier by a predetermined length of time than a time of occurrence of a predetermined state change in the subjects. The generating unit is configured to generate, on the basis of information about the medical data in the specific time period, learning-purpose data used for generating a learned model configured to output information about the predetermined state change in a subject being subject to a prediction that may occur at a time later by the predetermined length of time than a time at which a medical service is provided for the subject.

Exemplary embodiments of a medical information processing apparatus will be explained in detail below, with reference to the accompanying drawings. In the embodiments below, examples will be explained in which the subject is a patient.

### First Embodiment

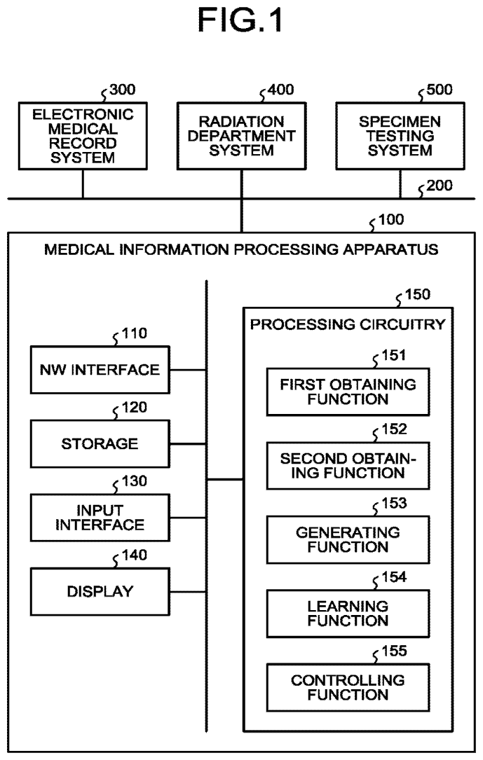

FIG. 1 is a diagram illustrating an exemplary configuration of a medical information processing apparatus according to a first embodiment.

For example, as illustrated in FIG. 1, a medical information processing apparatus 100 according to the present embodiment is communicably connected to an electronic medical record system 300, a radiation department system 400, and a specimen testing system 500, and the like via a network 200. For example, the medical information processing apparatus 100 and the systems are installed in a hospital or the like and are connected to one another via the network 200, which is an intra-hospital Local Area Network (LAN) or the like.

The electronic medical record system 300 is a system configured to generate and manage medical data related to prescriptions, nursing records, and the like provided for patients. The radiation department system 400 is a system configured to generate and manage medical data related to vital and imaging tests and the like performed on patients. The specimen testing system 500 is a system configured to generate and manage medical data related to specimen tests and the like performed on patients. In the present example, every time a medical service is provided for a patient, each of the systems generates a new piece of medical data and transmits the generated piece of medical data to the medical information processing apparatus 100. For example, every time any of the systems generates a new piece of medical data, the system transmits the generated piece of medical data to the medical information processing apparatus 100 either in response to a request from the medical information processing apparatus 100 or autonomously.

The medical information processing apparatus 100 is configured to obtain various types of medical data from the electronic medical record system 300, the radiation department system 400, and the specimen testing system 500 via the network 200 and to perform various types of information processing processes by using the obtained medical data. For example, the medical information processing apparatus 100 is realized by using a computer device such as a workstation, a personal computer, a tablet terminal, or the like.

More specifically, the medical information processing apparatus 100 includes a network (NW) interface 110, a storage 120, an input interface 130, a display 140, and processing circuitry 150.

The NW interface 110 is connected to the processing circuitry 150 and is configured to control transfer and communication of various types of data between the medical information processing apparatus 100 and the systems. More specifically, the NW interface 110 is configured to receive the medical data from each of the systems and to output the received medical data to the processing circuitry 150. For example, the NW interface 110 is realized by using a network card, a network adaptor, a Network Interface Controller (NIC), or the like.

The storage 120 is connected to the processing circuitry 150 and is configured to store therein various types of data. More specifically, the storage 120 is configured to store therein the medical data received from the systems. For example, the storage 120 is realized by using a semiconductor memory element such as a Random Access Memory (RAM) or a flash memory, or a hard dick, an optical disk, or the like.

The input interface 130 is connected to the processing circuitry 150 and is configured to receive operations to input various types of instructions and various types of information from an operator. More specifically, the input interface 130 is configured to convert the input operations received from the operator into electric signals and to output the electric signals to the processing circuitry 150. For example, the input interface 130 is realized by using a trackball, a switch button, a mouse, a keyboard, a touchpad on which an input operation is performed by touching the operation surface thereof, a touch screen in which a display screen and a touchpad are integrally formed, and/or a contactless input circuit using an optical sensor, and an audio input circuit or the like. In the present disclosure, the input interface 130 does not necessarily have to include one or more physical operation component parts such as a mouse and a keyboard. For instance, possible examples of the input interface 130 include an electrical signal processing circuit configured to receive an electrical signal corresponding to an input operation from an external input device provided separately from the apparatus and to output the electrical signal to a controlling circuit.

The display 140 is connected to the processing circuitry 150 and is configured to display various types of information and various types of images. More specifically, the display 140 is configured to convert data of various types of information and various types of images sent thereto from the processing circuitry 150, into display-purpose electrical signals and to output the electrical signals. For example, the display 140 is realized by using a liquid crystal monitor, a Cathode Ray Tube (CRT) monitor, a touch panel, or the like. The display 140 is an example of a display unit.

The processing circuitry 150 is configured to control constituent elements of the medical information processing apparatus 100 in response to the input operations received from the operator via the input interface 130. More specifically, the processing circuitry 150 is configured to store the medical data output from the NW interface 110 into the storage 120. Further, the processing circuitry 150 is configured to read the medical data from the storage 120 and to cause the display 140 to display the read medical data.

An overall configuration of the medical information processing apparatus 100 according to the present embodiment has thus been explained. The medical information processing apparatus 100 according to the present embodiment configured as described above has a function of predicting an advance indication of a predetermined state change that may occur to the patient. In this situation, the predetermined state change denotes, for example, an unwanted state change or an undesirable state change from a clinical viewpoint. Specific examples of the predetermined state change include, for instance, a sudden change in the clinical state, occurrence of a side effect of treatment, and deterioration of the clinical state (e.g., a postoperative infection) caused by a factor that is not directly related to a treated disease.

For example, as an example of such a function, a technique for automatically monitoring degradation in hemodynamics has been proposed by which a vital signs instability index (VIX) threshold value for a patient is calculated on the basis of VIX values from a predetermined time period in the past for the purpose of avoiding getting too many false alarms from erroneous detection, so as to determine that the state of the patient is deteriorating when the VIX threshold value exceeds a predetermined percentage. According to this technique, a model used for calculating the VIX values may be a logistic regression model, for example, and is derived from various patient subpopulations on the basis of various parameters.

However, in such a model used for predicting deterioration in the clinical state, because vital signs and specimen test data are directly used as inputs, erroneous detection may occur when medical data having many outliers such as pediatric medical data, for example, is used. Further, in such a model used for predicting deterioration in the clinical state, the threshold value used for determining the deterioration in the clinical state is calculated from times in the past; however, because it is not taken into consideration whether or not the data used for calculating the threshold value is data from normal time, it may not be possible, in some situations, to correctly judge deterioration in the clinical state.

To cope with these situations, the medical information processing apparatus 100 according to the present embodiment is configured to be able to more accurately predict an advance indication of the predetermined state change in the patient. In the present embodiment, an example will be explained in which the predetermined state change denotes a sudden change in the clinical state.

More specifically, the medical information processing apparatus 100 predicts the advance indication of a sudden change in the clinical state, by using an already-learned model (hereinafter “learned model”) configured to receive an input of information about medical data at or earlier than a time at which a medical service is provided for a patient subject to the prediction and to output information about a sudden change in the clinical state of the patient that may occur at a time later by a predetermined length of time than the time of the medical service.

In the present embodiment, the medical information processing apparatus 100 constructs the learned model (a prediction model) configured to receive an input of information about medical data in a specific time period at or earlier than the time at which the medical service is provided for the patient subject to the prediction and to output a probability of the occurrence of a sudden change in the clinical state of the patient. Further, every time a medical service is provided for the patient, the medical information processing apparatus 100 predicts the occurrence of a sudden change in the clinical state of the patient, by using the constructed learned model. In the present embodiment, the information input to the learned model is statistical feature values calculated from the medical data in the specific time period mentioned above.

With this configuration, according to the present embodiment, because the statistical feature values calculated from the medical data in the specific time period are used, it is possible to more accurately predict an advance indication of a sudden change in the clinical state such as heart failure, even when data having many outliers such as pediatric medical data, for example, is used. In the following sections, a configuration of the medical information processing apparatus 100 structured as described above will be explained in detail.

In the present embodiment, an example will be explained in which the medical information processing apparatus 100 predicts the occurrence of a sudden change in the clinical state of the patient in units of days. In other words, in the present embodiment, the example will be explained in which the learned model is configured to receive an input of information about medical data in a target time period on or earlier than the day on which a medical service is provided for the patient subject to the prediction and is configured to output information about a sudden change in the clinical state of the patient that may occur at a time later by a predetermined number of days than the day of the medical service Further, in the present embodiment, the example will be explained in which the information about the occurrence of a sudden change in the clinical state is a probability of the occurrence of a sudden change in the clinical state.

First, in the present embodiment, the storage 120 has stored therein the learned model having a function of receiving an input of statistical feature values in the target time period on or earlier than the day on which the medical service is provided for the patient subject to the prediction and of outputting the probability of a sudden change in the clinical state of the patient that may occur at a time later by the predetermined number of days than the day of the medical service. The storage 120 is an example of a storage unit.

Further, in the present embodiment, the processing circuitry 150 includes a first obtaining function 151, a second obtaining function 152, a generating function 153, a learning function 154, and a controlling function 155. The first obtaining function 151 is an example of a first obtaining unit. The second obtaining function 152 is an example of a second obtaining unit and the obtaining unit. The generating function 153 is an example of the generating unit. The learning function 154 is an example of the learning unit. The controlling function 155 is an example of the controlling unit.

The first obtaining function 151 is configured to obtain medical data related to patients.

More specifically, every time a new piece of medical data is generated by any of the electronic medical record system 300, the radiation department system 400, and the specimen testing system 500, the first obtaining function 151 obtains the generated piece of medical data from the system and stores the obtained piece of medical data into the storage 120. As a result, pieces of medical data generated by the electronic medical record system 300, the radiation department system 400, and the specimen testing system 500 are added to the storage 120 from time to time and accumulated.

Further, in the present embodiment, the processing circuitry 150 performs processes at the time of learning (hereinafter “learning mode”) to generate the learned model and processes at the time of operation (hereinafter, “operation mode”) to use the generated learned model. In this situation, for example, the processing circuitry 150 performs the processes in the learning mode either with predetermined timing (e.g., regularly at predetermined time intervals) or at a time when an instruction is received from the operator indicating that a learning process should be started. Further, for example, the processing circuitry 150 performs the processes in the operation mode when a new piece of medical data is obtained from any of the electronic medical record system 300, the radiation department system 400, and the specimen testing system 500.

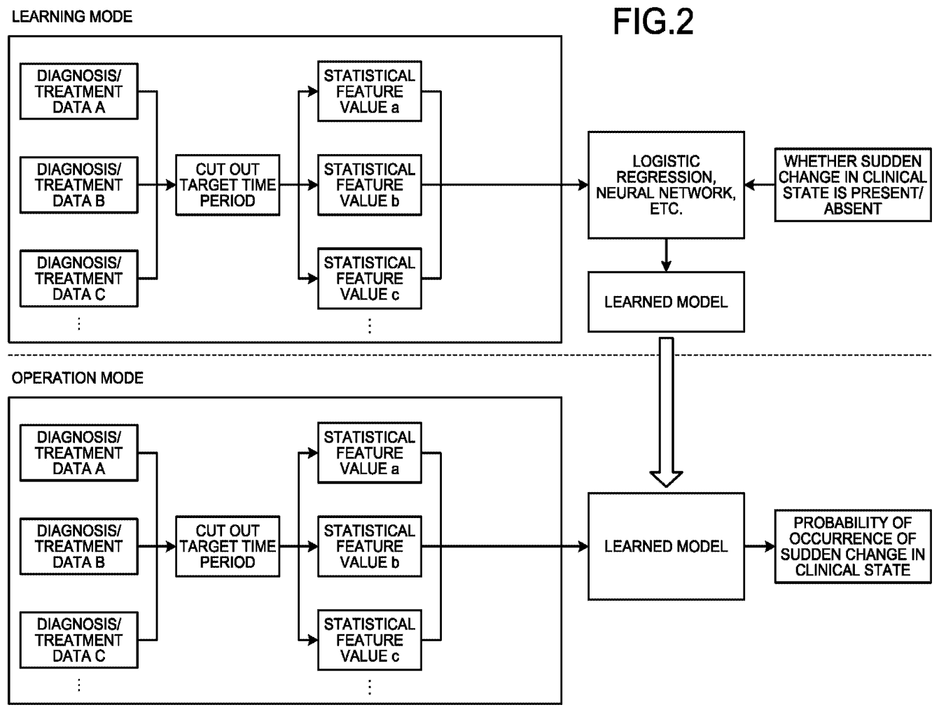

FIG. 2 is a chart illustrating processes performed in the learning mode and the operation mode by the processing circuitry 150 according to the first embodiment.

First, the processes in the learning mode will be explained.

In the learning mode, among medical data related to patients, the second obtaining function 152 obtains medical data in a specific time period at or earlier than a previous point in time that is earlier by a predetermined length of time than a time of the occurrence of a sudden change in the clinical state.

For example, as illustrated in the top section of FIG. 2, in the learning mode, the second obtaining function 152 obtains sudden change information indicating whether a sudden change in the clinical state is present or absent in the patients and obtains, from among the medical data related to the patients, medical data in the target time period on or earlier than a previous date that is earlier by a predetermined number of days than the day of the occurrence of the sudden change in the clinical state.

More specifically, the second obtaining function 152 obtains the medical data of a plurality of patients accumulated in the storage 120 either with predetermined timing or at a time when an Instruction is received from the operator indicating that a learning process should be started. Further, second obtaining function 152 obtains the sudden change information indicating whether a sudden change in the clinical state is present or absent in the patients whose medical data was obtained. In this situation, the sudden change information is stored in the storage 120 for each of the patients and is updated by the controlling function 155 (explained later) when a sudden change in the clinical state has occurred to any of the patients.

Further, on the basis of the information about the medical data in the specific time period obtained by the second obtaining function 152, the generating function 153 generates learning-purpose data used for generating the learned model configured to receive an input of the information about the medical data in the specific time period at or earlier than the time at which a medical service is provided for the patient subject to the prediction and configured to output the information about a sudden change in the clinical state of the patient that may occur at a time later by the predetermined length of time than the time of the medical service.

For example, the generating function 153 generates data including the statistical feature values calculated from the medical data in the target time period and the sudden change information, as the learning-purpose data used for generating the learned model configured to receive an input of the statistical feature values in the target time period on or earlier than the day on which the medical service is provided for the patient subject to the prediction and configured to output a probability of a sudden change in the clinical state of the patient that may occur at a time later by the predetermined number of days than the day of the medical service.

More specifically, from the various types of medical data (pieces of medical data A, B, C and so on illustrated in FIG. 2) in the target time period obtained by the second obtaining function 152, the generating function 153 calculates various types of statistical feature values (statistical feature values a, b, c, and so on illustrated in FIG. 2) in correspondence with the various types of medical data. After that, the generating function 153 generates, as the learning-purpose data, data in which the calculated various types of statistical feature values are kept in correspondence with the sudden change information obtained by the second obtaining function 152, for each patient.

In this situation, for example, the medical data may include vital sign measured values, observation item measured values, specimen test measured values, image measured values, and the like. In this situation, the vital sign measured values may include, for example, a pulse rate, blood pressure levels (systolic blood pressure, diastolic blood pressure, average blood pressure), a respiration rate, body temperature, SpO₂ (arterial blood oxygen saturation), and the like. The observation item measured values may include the weight of the patient, a volume of urine, In/Out balance (a fluid intake amount and a fluid excretion amount or the difference of the two), and the like. The specimen test measured values may include, for example, hemoglobin (Hb), Brain Natriuretic Peptide (BNP), N-terminal pro-Brain Natriuretic Peptide (NTproBNP), Human Atrial Natriuretic Peptide (HAND), Blood Urea Nitrogen (BUN), serum creatinine (Cre), lactate (Lac), Base Excess (BE), pO₂ (oxygen partial pressure), pCO₂ (carbon dioxide partial pressure), hydrogen ion concentration (pH), and the like. The image measured values may include, for example, a Cardio-Thoracic Ratio (CTR) from a chest X-ray image, Left Ventricular Ejection Fraction (LVEF) from an echocardiogram, and the like.

Further, for example, as a category variable used in machine learning processes, medical data expressed without using numerical values may further be added to the learning-purpose data. For example, the medical data expressed without using numerical values may include information about a background of the patient, an intervention status, a score, rules, and the like. The information about the background of the patient may include, for example, the age, the gender, and the name of the disease of the patient. Further, the information about the intervention status may include, for example, information about medication, respiratory management, and the like. The information about the score may be, for example, a National Early Warning Score (NEWS). The information about the rules may be, for example, control chart abnormality judgment rules used for quality control, or the like.

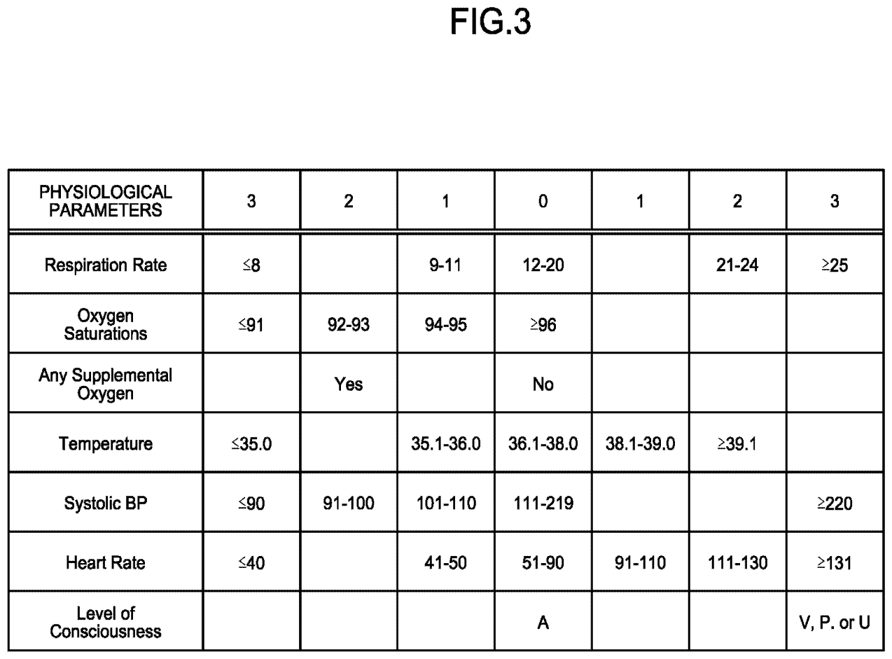

FIG. 3 is a table of the NEWS used as an example of the medical data according to the first embodiment.

For example, as illustrated in FIG. 3, the NEWS include scores indicating levels of risk occurring to a patient and is defined with four levels of 0 to 3 according to values of seven types of physiological parameters such as a respiration rate, an oxygen saturation level, the presence/absence of any supplemental oxygen, the body temperature, a systolic blood pressure (BP) level, a heart rate, and a level of consciousness.

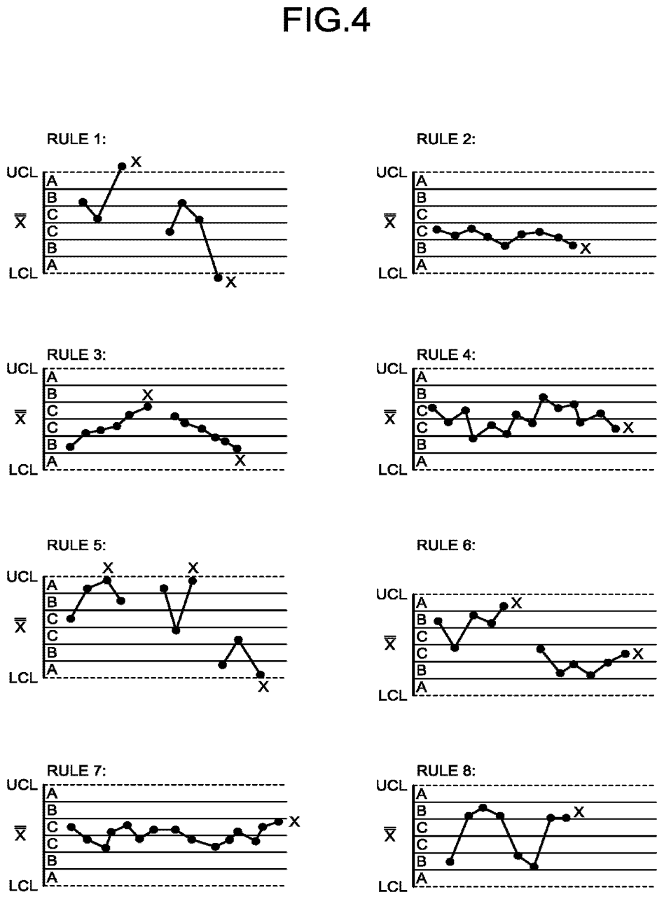

FIG. 4 presents charts illustrating examples of control chart abnormality judgement rules used as another example of the medical data according to the first embodiment.

For example, as illustrated in FIG. 4, the control chart abnormality judgment rules are rules used for judging abnormalities in a plurality of consecutive data values. Each of a plurality of patterns indicating distributions of data values considered abnormal is defined as a rule.

For instance, in the example in FIG. 4, Rule 1 is a pattern in which one data value is beyond region A (out of control). Rule 2 is a pattern in which nine data values are on the same side with respect to the center line (consecutive). Rule 3 is a pattern in which six data values increase or decrease (a rising trend/a falling trend). Rule 4 is a pattern in which fourteen data values increase and decrease alternately. Rule 5 is a pattern in which, of three consecutive data values, two data values are in region A or a region beyond (being close to a control border line). Rule 6 is a pattern in which, of five consecutive points, four points are in region B or a region beyond (a centralization trend). Rule 7 is a pattern in which fifteen consecutive points are in region C (a centralization trend). Rule 8 is a pattern in which eight consecutive points are in a region beyond region C. According to the control chart abnormality judgment rules, when a distribution of data values matches any of the plurality of patterns, it is determined that an abnormality is exhibited.

Further, the statistical feature values calculated from the aforementioned various types of medical data may include, for example, a feature value expressing a central tendency such as an average value, a median value, or a most frequent value, or a maximum value, a minimum value, or the like. Further, the statistical feature values may include, for example, a feature value expressing dispersion such as a variance, a standard deviation, an interquartile range, a range indicated with the length of a whisker when a distribution of data values is expressed with a box-and-whisker plot while excluding outliers, or a coefficient of variation. Further, the statistical feature values may include, for example, a feature value expressing a distribution such as a degree of kurtosis or a degree of skewness. Further, the statistical feature values may include, for example, a statistical value indicating a relationship between pieces of data such as a covariance, a correlational coefficient, or autocorrelation. Further, the statistical feature values may include, for example, a feature value related to frequencies such as a specific frequency component of a power spectrum. Further, for example, the statistical feature values may be related to a nursing record such as levels defining, at different stages, health conditions of the patient being good or bad.

Alternatively, the statistical feature values may include a feature value calculated on the basis of any of the feature values described above. For example, the statistical feature values may include a feature value expressing a temporal change such as a change amount, a change ratio, or a second-order difference. Further, the statistical feature values may include, for example, a feature value expressing a trend in temporal changes such as a simple moving average, a weighted moving average, an exponential moving average, or the like.

Further, the learning function 154 is configured to generate the aforementioned learned model by performing a machine learning process while using, as the learning-purpose data, the information about the medical data at or earlier than the time at which a medical service is provided for a patient and the information about occurrence of the predetermined state change in the patient.

For example, the learning function 154 generates the aforementioned learned model by performing the machine learning process while using, as the learning-purpose data, the statistical feature values calculated from the medical data in the target time period related to the patients and the sudden change information indicating whether a sudden change in the clinical state is present or absent in the patient.

More specifically, the learning function 154 generates the learned model by performing the machine learning process based on an algorithm such as a logistic regression, a neural network, or the like, while using the learning-purpose data generated by the generating function 253. After that, the learning function 154 stores the generated learned model into the storage 120. At that time, when the storage 120 has already stored therein a learned model previously generated, the learning function 154 replaces the stored learned model with the newly-generated learned model.

In this situation, a specific example of the processes performed in the learning mode by the processing circuitry 150 according to the present embodiment will be explained. In the following sections, an example will he explained in which the predetermined number of days is six days, so that a probability of the occurrence of a sudden change in the clinical state is predicted six days earlier than the occurrence of the sudden change in the clinical state. In that situation, the learning function 154 generates a learned model having the function of outputting a probability of a sudden change in the clinical state of the patient that may occur six days later than the day on which the medical service is provided for the patient subject to the prediction.

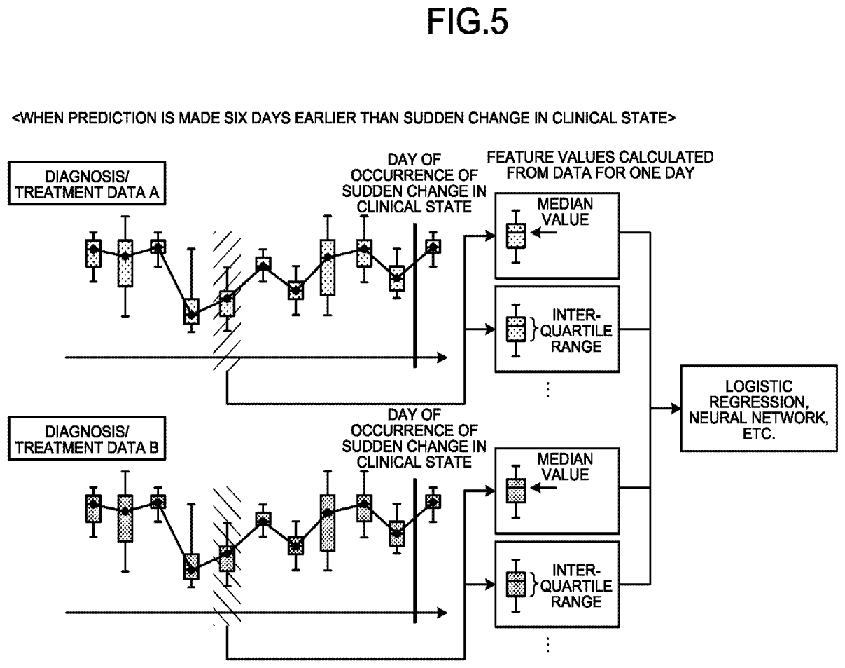

FIG. 5 is a chart illustrating a first example related to processes in the learning mode performed by the processing circuitry 150 according to the first embodiment.

The first example is an example in which the target time period is a certain time period included in the day that is six days earlier than the day of the occurrence of a sudden change in clinical state. In the present example, the certain time period is one day; however, the certain time period does not necessarily have to be one day and may be twelve hours or one week.

In this situation, for example, as illustrated in FIG. 5, with respect to medical data A and medical data B of each patient, the second obtaining function 152 obtains medical data for one day of the day that is six days earlier than the day of the occurrence of a sudden change in the clinical state and sudden change information indicating whether a sudden change in the clinical state is present or absent in the patient.

Further, with respect to the medical data A and the medical data 5, the generating function 153 calculates various types of statistical feature values such as a median value, an interquartile range, and the like, from the medical data for the one day obtained by the second obtaining function 152. Further, the generating function 153 generates, as learning-purpose data, data in which the calculated statistical feature values are kept in correspondence with the sudden change information obtained by the second obtaining function 152, for each patient.

Further, the learning function 154 generates a learned model by performing a machine learning process based on an algorithm such as a logistic regression, a neural network, or the like, while using the learning-purpose data generated by the generating function 153.

As a result, the learned model is generated in the first example, the learned model being configured to receive the input of the statistical feature values for the one day of the day on which the medical service is provided for the patient subject to the prediction and configured to output a probability of a sudden change in the clinical state of the patient that may occur six days later than the day of the medical service.

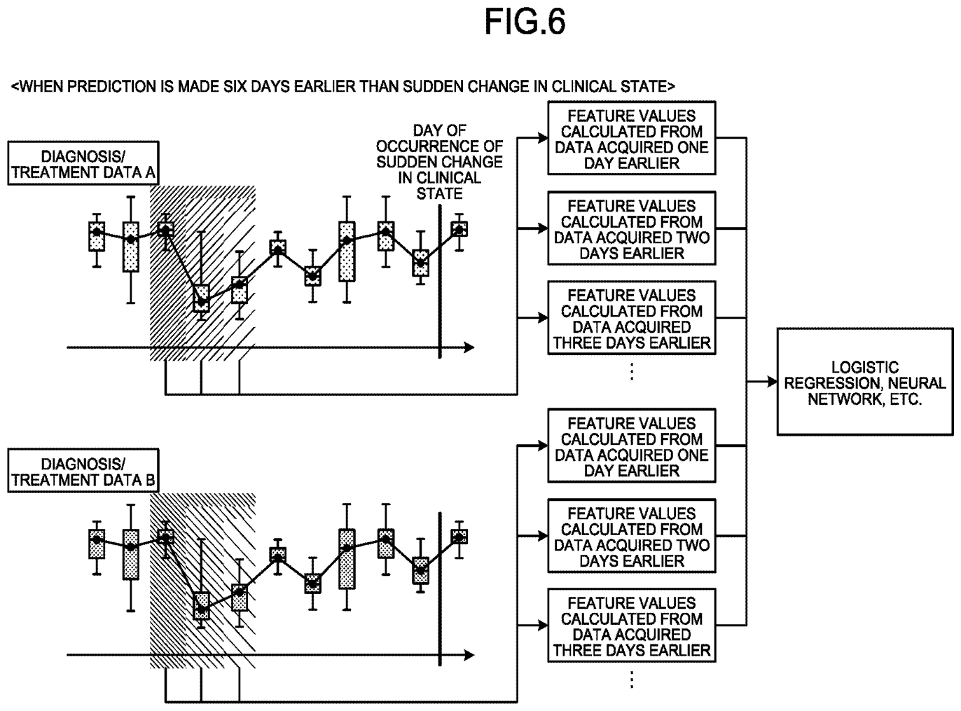

FIG. 6 is a chart illustrating a second example related to processes in the learning mode performed by the processing circuitry 150 according to the first embodiment.

In this situation, the second example is an example in which the target time period is represented by mutually-different time periods on or earlier than the previous date that is six days earlier than the day of the occurrence of the sudden change in the clinical state, the mutually-different time periods each including a certain time period on the previous date that is six days earlier than the day of the occurrence of the sudden change in the clinical state. In the present example, the certain time period is one day; however, the certain time period does not necessarily have to be one day and may be twelve hours or one week.

In this situation, for example, as illustrated in FIG. 6, with respect to the medical data A and the medical data B of each patient, the second obtaining function 152 obtains the medical data for one day of the previous date that is six days earlier than the day of the occurrence of the sudden change in the clinical state; the medical data for the two days including the previous date six days earlier and the day immediately preceding the previous date; the medical data for the three days including the previous date six days earlier, the day immediately preceding the previous date, and the day before the immediately-preceding day; and sudden change information indicating whether a sudden change in the clinical state is present or absent in the patient.

Further, with respect to the medical data A and the medical data B, the generating function 153 calculates various types of statistical feature values such as a median value, an interquartile range, and the like, from the medical data for the one day, the medical data for the two days, and the medical data for the three days obtained by the second obtaining function 152. Further, the generating function 155 generates, as learning-purpose data, data in which the calculated statistical feature values are kept in correspondence with the sudden change information obtained by the second obtaining function 152, for each patient.

After that, the learning function 154 generates a learned model by performing a machine learning process based on an algorithm such as a logistic regression, a neural network, or the like, while using the learning-purpose data generated by the generating function 153.

As a result, the learned model is generated in the second example, the learned model being configured to receive the input of the statistical feature values for the one day of the day on which the medical service is provided for the patient subject to the prediction, the statistical feature values for the two days including the immediately-preceding day, and the statistical feature values for the three days further including the day before the immediately-preceding day and configured to output a probability of a sudden change in the clinical state of the patient that may occur six days later than the day of the medical service.

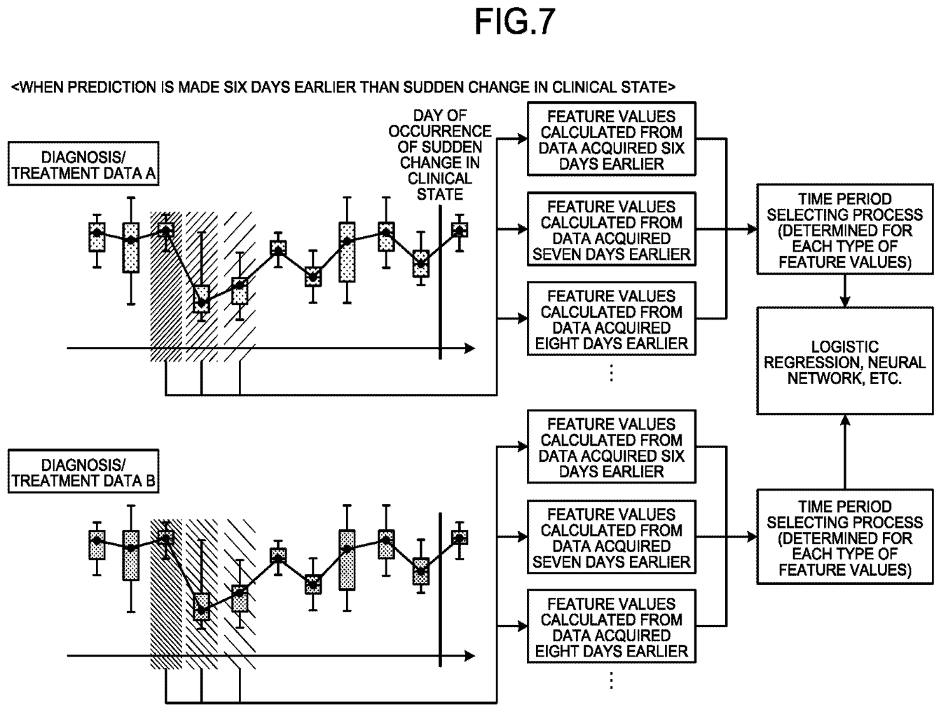

FIG. 7 is a chart illustrating a third example related to processes in the learning mode performed by the processing circuitry 150 according to the first embodiment.

The third example is an example in which the target time period is a time period selected in accordance with statistical feature values from among a plurality of mutually-different time periods on or earlier than the previous date that is six days earlier than the day of the occurrence of the sudden change in the clinical state. In the present example, the mutually-different time periods are each one day; however, the mutually-different time periods do not necessarily have to be one day and may he twelve hours or one week.

In this situation, for example, as illustrated in FIG. 7, with respect to the medical data A and the medical data B of each patient, the second obtaining function 152 obtains the medical data for one day of a previous date that is six days earlier than the day of the occurrence of the sudden change in the clinical state; the medical data for one day of another previous date seven days earlier; the medical data for one day of yet another previous date eight days earlier; and sudden change information indicating whether a sudden change in the clinical state is present or absent in the patient. In the present example, the situation is explained in which the pieces of medical data for the three consecutive days are obtained; however, the time periods of which pieces of medical data are obtained do not necessarily have to be consecutive and may be non-consecutive time periods.

Further, with respect to the medical data A and the medical data 5, the generating function 153 calculates various types of statistical feature values such as a median value, an interquartile range, and the like, from the medical data for the one day of the day six days earlier, the medical data for the one day of the day seven days earlier, and the medical data for the one day of the day eight days earlier obtained by the second obtaining function 152. After that, the generating function 153 selects one of the three time periods, for each of the calculated various types of statistical feature values. For example, the generating function 153 determines one of the three time periods having the largest statistical feature value as a selected time period. Further, the generating function 153 generates, as learning-purpose data, data in which the statistical feature values in the selected time period are kept in correspondence with the sudden change information obtained by the second obtaining function 152, for each patient.

Further, the learning function 154 generates a learned model by performing a machine learning process based on an algorithm such as a logistic egression, a neural network, or the like, while using the learning-purpose data generated by the generating function 153.

As a result, the learned model is generated in the third example, the learned model being configured to receive the input of the statistical feature values in the time period selected, in accordance with the statistical feature values, from among the one day of the day on which the medical service is provided for the patient subject to the prediction; the one day of the immediately-preceding day; and the one day of the day before the immediately-preceding day, and configured to output a probability of a sudden change in the clinical state of the patient that may occur six days later than the day of the medical service.

Next, returning to the description of FIG. 2, processes performed in the operation mode will be explained.

In the operation mode, the second obtaining function 152 obtains medical data at or earlier than the time at which the medical service is provided for a patient. More specifically, in the operation mode, the second obtaining function 152 obtains the medical data in a specific time period at or earlier than the time at which the medical service is provided for the patient.

For example, as illustrated in the bottom section of FIG. 2, in the operation mode, the second obtaining function 152 obtains the medical data in a target time period on or earlier than the day on which the medical service is provided for the patient.

More specifically, when a new piece of medical data is obtained by the first obtaining function 151, the second obtaining function 152 obtains the medical data in the target time period on or earlier than the day of the medical service indicated in the new piece of medical data, from among the medical data stored in the storage 120 with respect to the patient indicated in the new piece of medical data.

Further, the controlling function 155 exercises control by inputting the information about the medical data obtained by the second obtaining function 152 to the aforementioned learned model and causing the learned model to output information about a sudden change in the clinical state of the patient that may occur at a time later by a predetermined length of time than the time of the medical service.

For example, the controlling function 155 exercises control by inputting statistical feature values calculated from the medical data in the target time period on or earlier than the time at which the medical service is provided for the patient to the aforementioned learned model and causing the learned model to output a probability of a sudden change in the clinical state of the patient that may occur at the time later by the predetermined number of days than the day of the medical service.

More specifically, on the basis of the various types of medical data in the target time period obtained by the second obtaining function 152, the controlling function 155 calculates various types of statistical feature values in correspondence with the various types of medical data. After that, by inputting the calculated statistical feature values to the learned model stored in the storage 120, the controlling function 155 causes the learned model to output the probability of a sudden change in the clinical state of the patient indicated in the new piece of medical data that may occur at the time later by the predetermined number of days than the day of the medical service indicated in the new piece of medical data. Further, the controlling function 155 stores the probability output from the learned model into the storage 120 as a prediction result.

For example, when the learned model is generated as indicated in the first example in FIG. 5, so as to receive an input of the statistical feature values for one day of the day on which a medical service is provided for the patient subject to the prediction, the second obtaining function 152 obtains the medical data for the one day of the day of the medical service indicated in the new piece of medical data. In teat situation, the controlling function 155 calculates various types of statistical feature values such as a median value, an interquartile range, and the like, from the medical data for the one day obtained by the second obtaining function 152 and, by inputting the calculated statistical feature values to the learned model, the learned model is caused to output a probability of the occurrence of a sudden change in the clinical state

Further, for example, when the learned model is generated as indicated in the second example in FIG. 6, so as to receive an input of the statistical feature values for the one day of the day on which a medical service is provided for a patient subject to the prediction, the statistical feature values for the two days including the immediately-preceding day, and the statistical feature values for the three days further including the day before the immediately-preceding day, the second obtaining function 152 obtains the medical data for the one day of the day of the medical service indicated in the new piece of medical data, the medical data for the two days including the immediately-preceding day, and the medical data for the three days further including the day before the immediately-preceding day. In that situation, the generating function 153 calculates the various types of statistical feature values such as a median value, an interquartile range, and the like from the medical data for the one day, the medical data for the two days, and the medical data for the three days obtained by the second obtaining function 152 and, by inputting the calculated statistical feature values to the learned model, the learned model is caused to output a probability of the occurrence of a sudden change in the clinical state.

Further, for example, when the learned model is generated as indicated in the third example in FIG. 7, so as to receive an input of the statistical feature values in a time period selected, in accordance with the statistical feature values, from among the one day of the day on which a medical service is provided for a patient subject to the prediction, the one day on the immediately-preceding day, and the one day on the day before the immediately-preceding day, the second obtaining function 152 obtains the medical data for the one day of the day of the medical service indicated in the new piece of medical data, the medical data for the one day on the immediately-preceding day, and the medical data for the one day on the day before the immediately-preceding day. In that situation, the generating function 153 calculates the various types of statistical feature values such as a median value, an interquartile range, and the like from the medical data for the one day six days earlier, the medical data for the one day seven days earlier, and the medical data for the one day eight days earlier obtained by the second obtaining function 152 and, by inputting the calculated statistical feature values in the time period selected in accordance with the calculated statistical feature values to the learned model, the learned model is caused to output a probability of the occurrence of a sudden change in the clinical state.

After that, the controlling function 155 causes the display 140 to display information about the medical data at or earlier than the time at which the medical service is provided for the patient and information about a sudden change in the clinical state of the patient that may occur at a time later by a predetermined length of time than the time of the medical service.

For example, the controlling function 155 causes the display 140 to display the statistical feature values calculated from the medical data in the target time period on or earlier than the day on which the medical service is provided for the patient and the information indicating the probability of a sudden change in the clinical state of the patient that may occur at the time later by the predetermined number of days than the day of the medical service.

More specifically, when the probability of the occurrence of a sudden change in the clinical state is output from the learned model as a result of inputting the statistical feature values to the learned model, the controlling function 155 causes the display 140 to display the information indicating the probability and information about the statistical feature values in the target time period serving as basis of the probability.

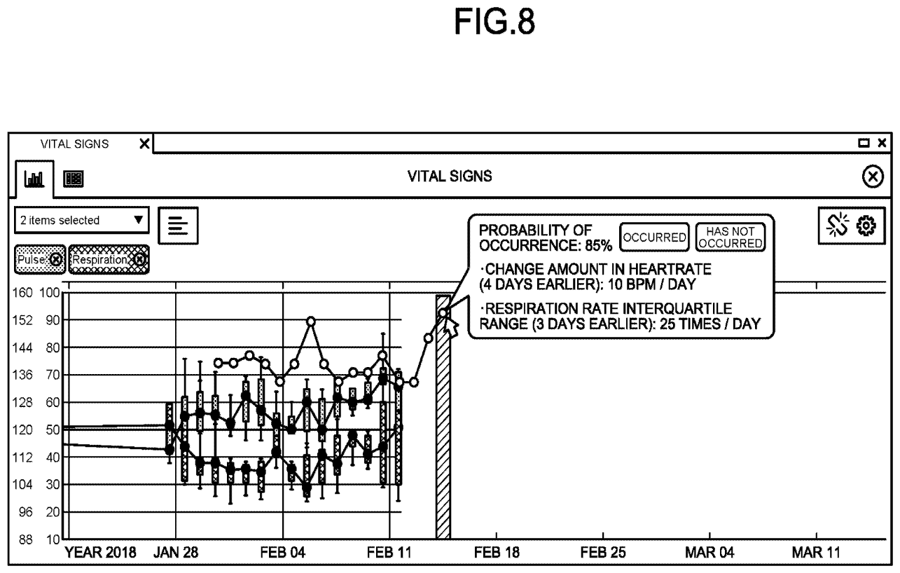

FIG. 8 is a drawing illustrating an example of a screen displayed on the display 140 by the controlling function 155 according to the first embodiment.

For example, as illustrated in FIG. 8, the controlling function 155 causes the display 140 to display information (“PROBABILITY OF OCCURRENCE: 85%” in FIG. 8) indicating a probability of the occurrence of a sudden change in the clinical state and information (the line graph plotted with black dots in FIG. 8) indicating chronological trajectories of the statistical feature values (“Pulse” and “Respiration” in FIG. 8) in the target time period serving as basis of the probability. In this situation, for example, when a time period is selected in accordance with the statistical feature values like in the third example in FIG. 7, the time periods may vary among the different types of statistical feature values. In that situation, for example, the controlling function 155 arranges the statistical feature values to be displayed in an overlapping manner by shifting the positions thereof so as to align the displayed time periods.

FIG. 8 illustrates the example in which, with respect to the statistical feature values serving as the basis of the probability of the occurrence of a sudden change in the clinical state, displayed is information indicating chronological trajectories not only for the target time period but also for the time periods other than the target time period. In that situation, for example, within the displayed information, the controlling function 155 displays the information about the statistical feature values in the target time period in a highlighted manner.

Further, FIG. 8 illustrates the example in which the statistical feature values are median values so that the line graph indicating the median values is displayed as the information indicating the chronological changes of the statistical feature values. However, for example, when the statistical feature values are not median values, it may not be possible to express the chronological changes with a line graph, in some situations. In those situations, the information indicating chronological changes in the statistical feature values may be displayed by using certain forms other than line graphs. For example, the statistical feature values may be displayed by using boxes in a box-and-whisker plot, like the one displayed together with the line graph in FIG. 8.

Further, by referring to prediction results stored in the storage 120, the controlling function 155 displays information (a line graph plotted with white dots in FIG. 8) indicating chronological trajectories of the probability of the occurrence of a sudden change in the clinical state. In this situation, for example, the controlling function 155 refers to the prediction results stored in the storage 120 and displays information indicating a probability of a sudden change in the clinical state that may occur on a day that is later by a predetermined number of days than today. Alternatively, the controlling function 155 may display information indicating a probability of a sudden change in the clinical state that may occur today. Further, for example, when the most up-to-date probability exceeds a predetermined threshold value, the controlling function 155 displays the probability in a highlighted manner.

Further, with respect to the probability output from the learned model, the controlling function 155 receives, from the operator, an operation to input information indicating whether or not a sudden change in the clinical state actually occurred and arranges the input result to be fed back to the sudden change information.

For example, together with the information indicating the probability of the occurrence of a sudden change in the clinical state, the controlling function 155 displays a button used for receiving a notice that a sudden change in the clinical state occurred (“OCCURRED” in FIG. 8) and another button used for receiving a notice that a sudden change in the clinical state did not occur (“HAS NOT OCCURRED” in FIG. 8) and further receives an operation to select one of the buttons from the operator. After that, the controlling function 155 arranges the received result to be reflected in the sudden change information stored in the storage 120.

In this situation, for example, the controlling function 155 may judge whether or not a sudden change in the clinical state actually occurred on the basis of medical data of the patient and may arrange the judgment result to be fed back to the sudden change information. For example, the controlling function 155 may refer to the medical data stored in the storage 120 and, when it is identified that a treatment intervention is scheduled or emergency treatment (e.g., surgery) was provided for the patient, the controlling function 155 determines that a sudden change in the clinical state actually occurred.

In this situation, for example, the controlling function 155 displays a list view arranged with pieces of information obtained by simplifying the information contained in the screen illustrated in FIG. 8 with respect to a plurality of patients. Further, when an operation to designate one of the patients from the displayed list is received from the operator, the controlling function 155 displays the screen illustrated in FIG. 8 with respect to the designated patient. In that situation, as for the simplified information, for example, the controlling function 155 may display information by narrowing down the information to only such patients whose probability of the occurrence of a sudden change in the clinical state is equal to or higher than a threshold value or may display information about all the patients while highlighting the information about such patients whose probability of the occurrence of a sudden change in the clinical state is equal to or higher than the threshold value, by appending a flag or the like to the information about such patients.

The processing functions of the processing circuitry 150 have thus been explained. For example, the processing circuitry 150 may be realized by using a processor. In that situation, the processing functions of the processing circuitry 150 are stored in the storage 120 in the form of computer-executable programs. Further, the processing circuitry 150 realizes the functions corresponding to the programs by reading and executing the programs from the storage 120. In other words, the processing circuitry 150 that has read the programs has the functions illustrated within the processing circuitry 150 in FIG. 1. With reference to FIG. 1, the example was explained in which the processing functions are realized by the single processor; however, another arrangement is also acceptable in which processing circuitry is structured by combining together a plurality of independent processors, so that the functions are realized as a result of the processors executing the programs. Further, the processing functions of the processing circuitry 150 may be realized as being distributed to a plurality of processing circuits or as being integrated together into a single processing circuit, as appropriate. Further, with reference to FIG. 1, the example was explained in which the single storage (i.e., the storage 120) is configured to store therein the programs corresponding to the processing functions; however, another arrangement is also acceptable in which a plurality of storages are provided in a distributed manner, so that one or more processing circuitries read each of the corresponding programs from one of the individual storages.

For example, the process performed by the first obtaining function 151 to obtain the medical data from any of the electronic medical record system 300, the radiation department system 400, and the specimen testing system 500 is realized, for example, as a result of the processing circuitry 150 reading and executing a predetermined program corresponding to the first obtaining function 151 from the storage 120.

Further, for example, the processes performed in the learning mode and the operation mode by the second obtaining function 152, the generating function 153, the learning function 154, and the controlling function 155 are realized in the manner described below.

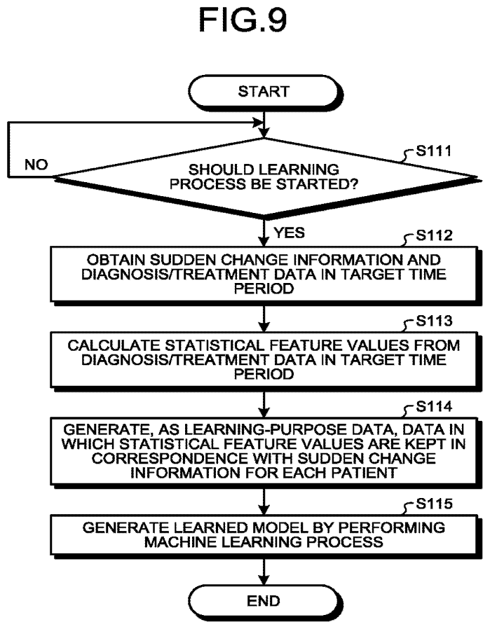

FIG. 9 is a flowchart illustrating a processing procedure of the processes in the learning mode performed by the medical information processing apparatus 100 according to the first embodiment.

For example, as illustrated in FIG. 9, in the learning mode, with predetermined timing or at a time when an instruction is received from the operator indicating that a learning process be started (step S111: Yes), the second obtaining function 152 refers to the storage 120 and obtains, with respect to a plurality of patients, sudden change information and medical data in a target time period (step S112).

After that, the generating function 153 calculates statistical feature values from the medical data in the target time period obtained by the second obtaining function 152 (step S113) and generates, as learning-purpose data, data in which the statistical feature values are kept in correspondence with the sudden change information for each of the patients (step S114).

Subsequently, the learning function 154 generates a learned model by performing a machine learning process while using the learning-purpose data generated by the generating function 153 (step S115).

In this situation, the processes at steps S111 and described above are realized, for example, as a result of the processing circuitry 150 reading and executing a predetermined program corresponding to the second obtaining function 152 from the storage 120. Further, the processes at steps S113 and S114 described above are realized, for example, as a result of the processing circuitry 150 reading and executing a predetermined program corresponding to the generating function 153 from the storage 120. Further, the process at steps S115 described above is realized, for example, as a result of the processing circuitry 150 reading and executing a predetermined program corresponding to the learning function 154 from the storage 120.

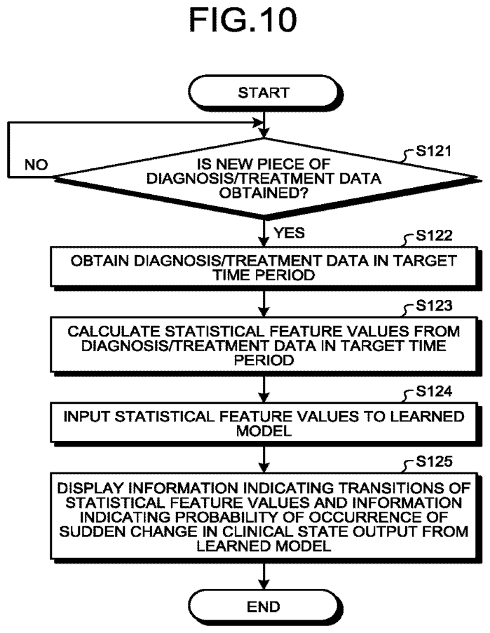

FIG. 10 is a flowchart illustrating a processing procedure of processes in the operation mode performed by the medical information processing apparatus 100 according to the first embodiment.

For example, as illustrated in FIG. 10, in the operation mode, when a new piece of medical data is obtained by the first obtaining function 151 (step S121: Yes), the second obtaining function 152 refers to the storage 120 and obtains the medical data in the target time period with respect to the patient indicated in the new piece of medical data (step S122).

After that, the controlling function 155 calculates statistical feature values from the medical data in the target time period obtained by the second obtaining function 152 (step S123) and by inputting the statistical feature values to the learned model stored in the storage 120, the controlling function 155 causes the learned model to output a probability of the occurrence of sudden change in the clinical state (step S124).

Subsequently, the controlling function 155 causes the display 140 to display information indicating transitions of the statistical feature values and information indicating the probability of the occurrence of a sudden change in the clinical state output by the earned model (step S125).

In this situation, the processes at steps S121 and S122 described above are realized, for example, as a result of the processing circuitry 150 reading and executing a predetermined program corresponding to the second obtaining function 152 from the storage 120. Further, the processes at steps S123 through S125 described above are realized, for example, as a result of the processing circuitry 150 reading and executing a predetermined program corresponding to the controlling function 155 from the storage 120.

As explained above, in the first embodiment, the first obtaining function 151 is configured to obtain the medical data related to the patient.

Further, in the learning mode, the second obtaining function 152 is configured to obtain the sudden change information indicating whether a sudden change in the clinical state is present or absent in the patients and obtain, from among the medical data related to the patients, the medical data in the target time period on or earlier than the previous date that is earlier by the predetermined number of days than the day of the occurrence of a sudden change in the clinical state. Further, the generating function 153 is configured to generate the data including the statistical feature values calculated from the medical data in the target time period and the sudden change information, as the learning-purpose data used for generating the learned model configured to receive the input of the statistical feature values in the target time period or earlier than the day on which the medical service is provided for the patient subject to the prediction and configured to output the probability of a sudden change in the clinical state of the patient that may occur at a time later by the predetermined number of days than the day of the medical service. Further, the learning function 154 is configured to generate the aforementioned learned model, by performing the machine learning process while using, as the learning-purpose data, the statistical feature values calculated from the medical data in the target time period related to the patients and the sudden change information indicating whether a sudden change in the clinical state is present or absent in the patients.

In contrast, in the operation mode, the second obtaining function 152 is configured to obtain the sudden change information indicating whether a sudden change in the clinical state is present or absent in the patients and to obtain, from among the medical data of the patients, medical data in the target time period on or earlier than the previous date that is earlier by the predetermined number of days than the day of the occurrence of a sudden change in the clinical state. Further, the controlling function 155 is configured to exercise control by inputting the statistical feature values calculated from the medical data in the target time period on or earlier than the day on which the medical service is provided for the patient to the aforementioned learned model and causing the learned model to output the probability of a sudden change in the clinical state of the patient that may occur at the time later by the predetermined number of days than the day of e medical service. Further, the controlling function 155 is configured to cause the display 140 to display the statistical feature values calculated from the medical data in the target time period on or earlier than the day on which the medical service is provided for the patient and the information indicating the probability of a sudden change in the clinical state of the patient that may occur at the time later by the predetermined number of days than the day of the medical service.

Consequently, according to the first embodiment, because the statistical feature values calculated from the medical data in the specific target time period are used, it is possible to more accurately predict an advance indication of a sudden change in the clinical state such as heart failure, even when data having many outliers such as pediatric medical data, for example, is used.

### Second Embodiment

In the first embodiment above, the example is explained in which the information input to the learned model is the statistical feature values calculated from the medical data in the specific time period at or earlier than the time at which the medical service is provided for the patient; however, possible embodiments are not limited to this example.

For example, the information input to the learned model may be relative values between the statistical feature values calculated from the medical data in a specific time period at or earlier than the time at which a medical service is provided for the patient and statistical feature values calculated from medical data in a reference time period determined for each patient.

For example, the information input to the learned model may be relative values between the statistical feature values calculated from the medical data in the target time period and the statistical feature values calculated from the medical data in the reference time period determined for each patient. In the following sections, this example will be explained as a second embodiment. The second embodiment will be explained while a focus is placed on differences from the first embodiment. Detailed explanations of some of the configurations that are the same as those in the first embodiment will be omitted.

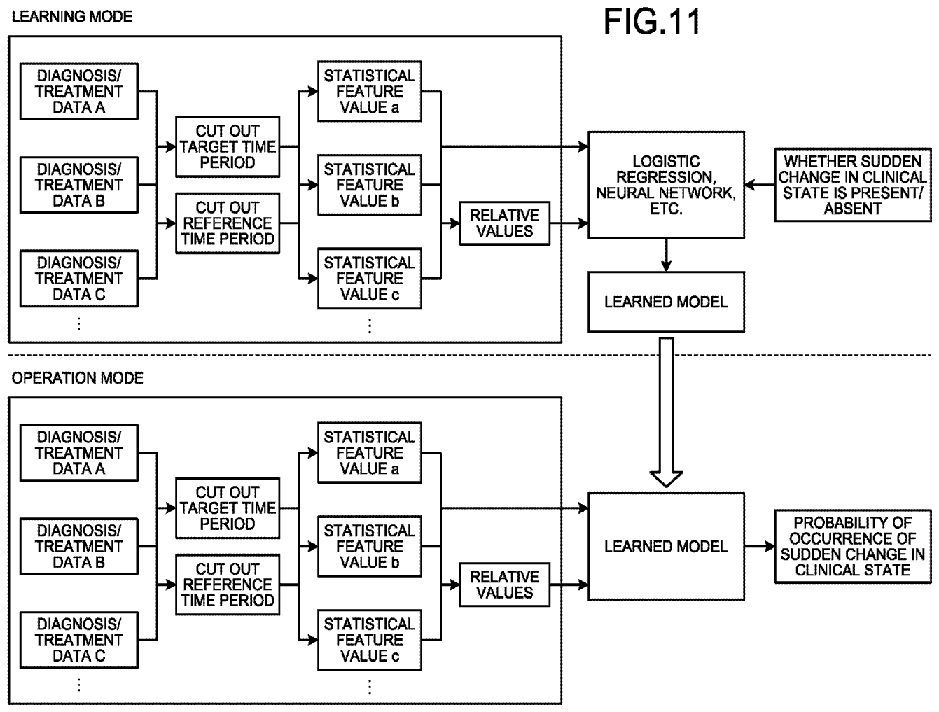

FIG. 11 is a chart illustrating processes in the learning mode and the operation mode performed by the processing circuitry 150 according to the second embodiment.

First, processes performed in the learning mode will be explained.

For example, as illustrated in the top section of FIG. 11, in the learning mode, the second obtaining function 152 obtains, similarly to the first embodiment, the sudden change information indicating whether a sudden change in the clinical state is present or absent in patients and to obtain, from among the medical data related to the patients, the medical data in the target time period on or earlier than a previous date that is earlier by a predetermined number of days than the day of the occurrence of the sudden change in the clinical state.

In this situation, in the second embodiment, the second obtaining function 152 is further configured to obtain the medical data in the reference time period determined for each patient, from among the medical data related to the patients.

More specifically, on the basis of the medical data accumulated in the storage 120, the second obtaining function 152 determines, as the reference time period, a time period exhibiting medical data corresponding to a reference value (a baseline value) specific to each patient and further obtains the medical data in the determined reference time period.

Further, the generating function 153 is configured to generate data including the relative values between the statistical feature values calculated from the medical data in the target time period and the statistical feature values calculated from the medical data in the reference time period as well as the sudden change information, as the learning-purpose data used for generating a learned model configured to receive an input of statistical feature values in the target time period on or earlier than the day on which a medical service is provided for a patient subject to the prediction and configured to output a probability of a sudden change in the clinical state of the patient that may occur at a time later by a predetermined number of days than the day of the medical service.

More specifically, from various types of medical data (medical data A, B, C, and so on illustrated in FIG. 11) in the target time period obtained by the second obtaining function 152, the generating function 153 calculates various types of statistical feature values (statistical feature values a, b, c, and so on illustrated in FIG. 11) in correspondence with the various types of medical data. Further, from various types of medical data (medical data A, B, C, and so on illustrated in FIG. 11) in the reference time period obtained by the second obtaining function 152, the generating function 153 calculates various types of statistical feature values (statistical feature values a, b, c, and so on illustrated in FIG. 11) in correspondence with the various types of medical data. After that, with respect to each type of statistical feature values, the generating function 153 calculates the relative values between the statistical feature values in the target time period and the statistical feature values in the reference time period and further generates, as the learning-purpose data, data in which the calculated relative values are kept in correspondence with the sudden change information obtained by the second obtaining function 152 for each patient.

After that, the learning function 154 generates the aforementioned learned model by performing a machine learning process while using, as the learning-purpose data, the relative values between the statistical feature values calculated from the medical data in the target time period related to the patients and the statistical feature values calculated from the medical data in the reference time period, as well as the sudden change information indicating whether a sudden change in the clinical state is present or absent in the patients.

Next, a specific example of the processes performed in the learning mode by the processing circuitry 150 according to the present embodiment will be explained. In the following sections, an example will be explained in which the predetermined number of days is six days, so that a probability of the occurrence of a sudden change in the clinical state predicted six days earlier than the occurrence of the sudden change in the clinical state. In that situation, the learning function 154 generates a learned model having the function of outputting the probability of a sudden change in the clinical state of the patient that may occur six days later than the day on which a medical service is provided for the patient subject to the prediction.

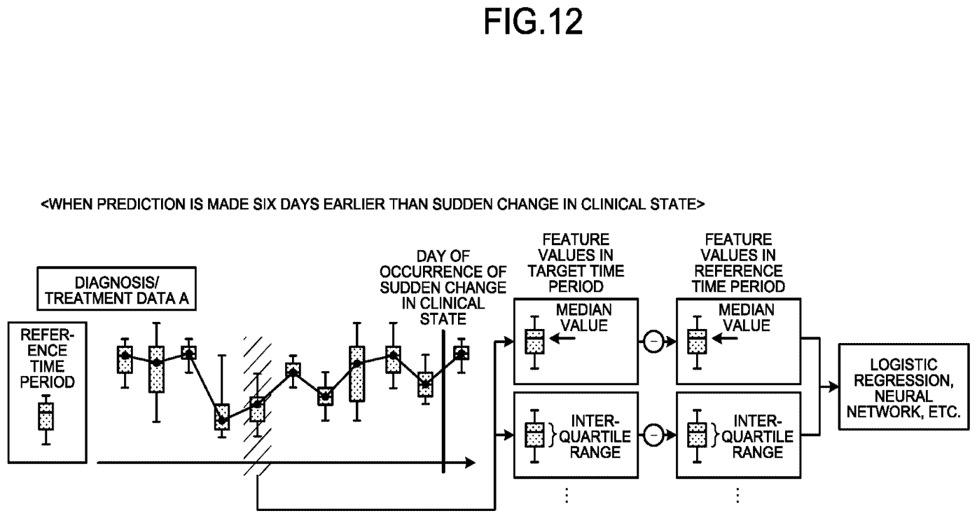

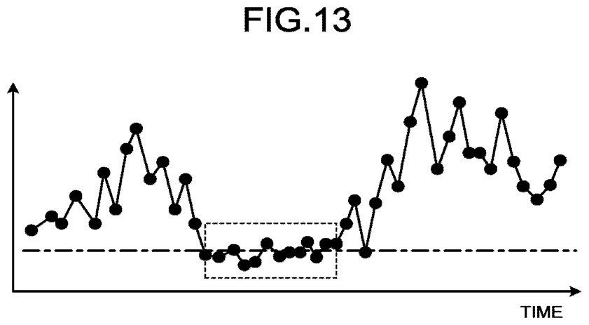

FIGS. 12 and 13 are charts illustrating examples related to the processes in the learning mode performed by the processing circuitry 150 according to the second embodiment.

In the present example, the target time period is a certain time period included in the day that is six days earlier than the day of the occurrence of a sudden change in the clinical state. In the present example, the certain time period is one day; however, the certain time period does not necessarily have to be one day and may be twelve hours or one week.

In the present example, for instance, as illustrated in FIG. 12, with respect to the medical data A related to the patients, the second obtaining function 152 obtains the medical data for one day of the previous date that is six days earlier than the day of the occurrence of a sudden change in the clinical state and the sudden change information indicating whether a sudden change in the clinical state is present or absent in the patients.

Also, with respect to the medical data A related to the patients, the second obtaining function 152 further determines a reference time period for each of the patients and obtains the medical data in the determined reference time period.

In this situation, for example, the reference time period is, as illustrated in FIG. 13, a time period in which the values of the medical data were most stable within a certain time period (the time period indicated with the dotted-line box in FIG. 13) in the past. For example, the reference time period may be a time period in which the fluctuation is the smallest in the feature values indicating a trend such as average values or median values or a time period in which feature values indicating dispersion such as variances or interquartile ranges are the smallest. For example, the reference time period may be a time period immediately preceding discharging of the patient from the hospital during the most recent hospitalization or a time period immediately following the hospitalization. Alternatively, when a trend is observed in long-term changes in the medical data, the reference time period may be, for example, a time period including an extrapolate value predicted on the basis of the trend.

Further, with respect to the medical data A, the generating function 153 calculates various types of statistical feature values such as a median value or an interquartile range, from the medical data in the target time period and the medical data in the reference time period obtained by the second obtaining function 152. After that, the generating function 153 calculates relative values between the statistical feature values calculated from the medical data in the target time period and the statistical feature values calculated from the medical data in the reference time period. For example, the generating function 153 calculates the relative values by performing a calculation such as subtraction, division, or the like. Further, the generating function 153 generates, as the learning-purpose data, the data in which the calculated relative values are kept in correspondence with the sudden change information obtained by the second obtaining function 152, for each patient.

After that, the learning function 154 generates the learned model by performing the machine learning process based on an algorithm such as a logistic regression, a neural network, or the like, while using the learning-purpose data generated by the generating function 153.

As a result, in the present example, the learned model has been generated, the learned model configured to receive an input of relative values between the statistical feature values for one day of the day on which a medical service is provided for a patient subject to the prediction and the statistical feature values calculated from the medical data in the reference time period determined for each patient and configured to output a probability of a sudden change in the clinical state of the patient that may occur six days later than the day of the medical service.

Next, returning to the description of FIG. 11, processes performed in the operation mode will be explained.

For example, as illustrated in the bottom section of FIG. 11, in the operation mode, the second obtaining function 152 obtains, similarly to the first embodiment, medical data in a target time period on or earlier than a day on which a medical service is provided for the patient.

In this situation, in the present embodiment, the second obtaining function 152 further obtains medical data in a reference time period determined for each patient, from among the medical data related to the patient.

Further, the controlling function 155 exercises control by inputting, to the aforementioned learned model, relative values between the statistical feature values calculated from the medical data in the target time period on or earlier than the day on which the medical service is provided for the patient and the statistical feature values calculated from the medical data in the reference time period and causing the learned model to output a probability of a sudden change in the clinical state of the patient that may occur at a time later by a predetermined number of days than the day of the medical service.

More specifically, from the various types of medical data in the target time period and the medical data in the reference time period obtained by the second obtaining function 152, the controlling function 155 calculates various types of statistical feature values in correspondence with the various types of medical data. Subsequently, the controlling function 155 calculates relative values between the statistical feature values in the target time period and the statistical feature values in the reference time period and, by inputting the calculated relative values to the learned model stored in the storage 120, the controlling function 155 causes the learned model to output a probability of a sudden change in the clinical state of the patient indicated in the new piece of medical data that may occur at a time later by the predetermined number of days than the day of the medical service indicated in the new piece of medical data. Further, the controlling function 155 stores the probability output from the learned model into the storage 120 as a prediction result.

For example, as illustrated in the example in FIG. 12, when the learned model is generated so as to receive an input of the relative values between the statistical feature values for one day of the day on which a medical service is provided for a patient subject to the prediction and the statistical feature values calculated from the medical data in the reference time period determined for each patient, the second obtaining function 152 obtains the medical data for one day of the day of the medical service indicated in the new piece of medical data and the medical data in the reference time period determined for each patient. In that situation, from the medical data for the one day and the medical data in the reference time period obtained by the second obtaining function 152, the controlling function 155 calculates various types of statistical feature values such as a median value, an interquartile range, and the like, and further, the controlling function 155 calculates the relative values of the feature values and, by inputting the calculated relative values to the learned model, causes the learned model to output a probability of the occurrence of a sudden change in the clinical state.

Further, similarly to the first embodiment, the controlling function 155 causes the display 140 to display the relative values calculated from the medical data in the target time period on or earlier than the day on which the medical service is provided for the patient and the information indicating the probability of a sudden change in the clinical state of the patient that may occur at a time later by the predetermined number of days than the day of the medical service.

For example, the processes in the learning mode and the processes in the operation mode performed by the second obtaining function 152, the generating function 153, the learning function 154, and the controlling function 155 may be realized in the manner described below.

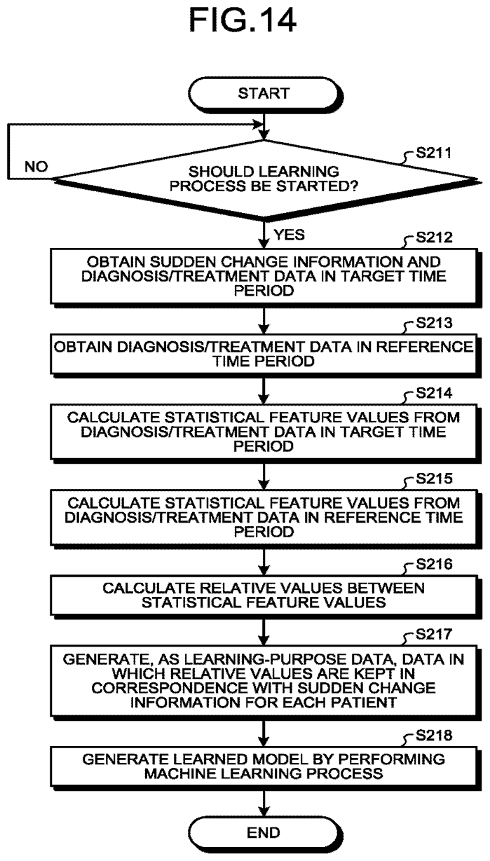

FIG. 14 is a flowchart illustrating a processing procedure of processes in the learning mode performed by the medical information processing apparatus 100 according to the second embodiment.

For example, as illustrated in FIG. 14, in the learning mode, with predetermined timing or at a time when an instruction is received from the operator indicating that a learning process be started (step S211: Yes), the second obtaining function 152 refers to the storage 125 and obtains, with respect to a plurality of patients, sudden change information and medical data in a target time period (step S212).

Further, for each of the patients, the second obtaining function 152 determines a reference time period on the basis of the medical data and further obtains the medical data in the reference time period (step S213).

After that, the generating function 153 calculates statistical feature values from the medical data in the target time period obtained by the second obtaining function 152 (step S214) and further calculates statistical feature values from the medical data in the reference time period (step S215).

Subsequently, the generating function 153 calculates relative values between the statistical feature values in the target time period and the statistical feature values in the reference time period (step S216) and generates, as learning-purpose data, data in which the calculated relative values are kept in correspondence with the sudden change information for each patient (step S217).

After that, the learning function 154 generates a learned model by performing a machine learning process while using the learning-purpose data generated by the generating function 153 (step S218).

In this situation, the processes at step S211 through S213 described above are realized, for example, as a result of the processing circuitry 150 reading and executing a predetermined program corresponding to the second obtaining function 152 from the storage 120. Further, the processes at step S214 through S217 described above are realized, for example, as a result of the processing circuitry 150 reading and executing a predetermined program corresponding to the generating function 153 from the storage 120. Furthermore, the process at step S218 described above is realized, for example, as a result of the processing circuitry 150 reading and executing a predetermined program corresponding to the learning function 154 from the storage 120.

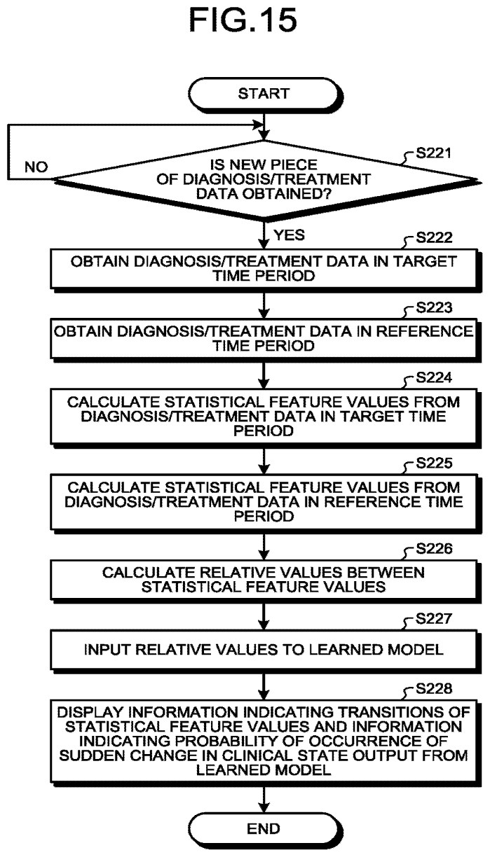

FIG. 15 is a flowchart illustrating a processing procedure of processes in the operation mode performed by the medical information processing apparatus 100 according to the second embodiment.

For example, as illustrated in FIG. 15, in the operation mode, when a new piece of medical data is obtained by the first obtaining function 151 (step S221: Yes), the second obtaining function 152 refers to the storage 120 and obtains medical data in the target time period with respect to the patient indicated in the new piece of medical data (step S222).

Further, with respect to the patient indicated in the new piece of medical data, the second obtaining function 152 determines a reference time period on the basis of the medical data and further obtains the medical data in the reference time period (step S223).

Subsequently, the controlling function 155 calculates statistical feature values from the medical data in the target time period obtained by the second obtaining function 152 (step S224) and further calculates statistical feature values from the medical data in the reference time period (step S225).

After that, the controlling function 155 calculates relative values between the statistical feature values in the target time period and the statistical feature values in the reference time period (step S226) and, by inputting the calculated relative values to the learned model stored in the storage 120, the controlling function 155 causes the learned model to output a probability of the occurrence of a sudden change in the clinical state (step S227).

Further, the controlling function 155 causes the display 140 to display information indicating transitions of the statistical feature values and information indicating the probability of the occurrence of a sudden change in the clinical state output from the learned model (step S228).

In this situation, the processes at steps S221 through S223 described above are realized, for example, as a result of the processing circuitry 150 reading and executing a predetermined program corresponding to the second obtaining function 152 from the storage 120. Further, the processes at step S224 through S228 described above are realized, for example, as a result of the processing circuitry 15C reading and executing a predetermined program corresponding to the controlling function 155 from the storage 120.

As explained above, in the second embodiment, the information input to the learned model is the relative values between the statistical feature values calculated from the medical data in the target time period and the statistical feature values calculated from the medical data in the reference time period determined for each patient.

Consequently, according to the second embodiment, even when reference values vary among the patients, it is possible to more accurately predict the advance indication of a sudden change in the clinical state such as heart failure.

### Other Embodiments

In the embodiments described above, the example is explained in which the medical information processing apparatus 100 is configured to predict the occurrence of a sudden change in the clinical state of the patient in units of days. However, possible embodiments are not limited to this example. For instance, the medical information processing apparatus 100 may be configured to predict the occurrence of a sudden change in the clinical state in units of hours.

In that situation, for example, the learned model is configured to receive an input of medical data in a specific time period expressed with hours as the medical data at or earlier than the time at which a medical service is provided for a patient subject to the prediction and is configured to output information about a sudden change in the clinical state of the patient that ay occur at a time later by a predetermined number of hours than the time of the medical service.

Further, in the learning mode, the second obtaining function 152 is configured to obtain medical data in a specific time period at or earlier than a previous point in time that is earlier by the predetermined number of hours than the time of the occurrence of a sudden change in the clinical state. In the operation mode, the second obtaining function 152 is configured to obtain medical data in a specific time period expressed with hours, as the medical data at or earlier than the time at which the medical service is provided for the patient.

In that situation, for example, in the operation mode, while shifting the time period of the medical data used for predicting the occurrence of a sudden change in the clinical state so as to partially overlap with each other, the medical information processing apparatus 100 may be configured to predict the occurrence of a sudden change in the clinical state at shorter time intervals than the time period.

More specifically, in that situation, the controlling function 155 predicts the occurrence of a sudden change in the clinical state by causing the learned model to output information about a sudden change in the clinical state of the patient at time intervals shorter than the specific time period for which the second obtaining function 152 obtains the medical data. For example, the controlling function 155 causes the learned model to output the information about the occurrence of a sudden change in the clinical state of the patient once every eight hours.

Further, every time the controlling function 155 predicts the occurrence of a sudden change in the clinical state, the second obtaining function 152 obtains medical data while shifting the time period for which medical data is obtained so that the time periods partially overlap with each other. For example, when the controlling function 155 predicts the occurrence of a sudden change in the clinical state once every eight hours, the second obtaining function 152 obtains the medical data by obtaining medical data corresponding to 24 hours while arranging pieces of medical data to overlap with each other by 16 hours.

Further, in the embodiments described above, the example is explained in which the information about the occurrence of a sudden change in the clinical state is the probability of the occurrence of a sudden change in the clinical state; however, possible embodiments are not limited to this example.

For instance, the information about the occurrence of a sudden change in the clinical state may be information indicating whether a sudden change in the clinical state is present or absent.

In that situation, for example, after deriving a probability of the occurrence of a sudden change in the clinical state on the basis of the medical data input thereto, the learned model judges whether a sudden change in the clinical state is present or absent according to the level of the derived probability and further outputs the judgment result. After that, on the basis of the judgment result output from the learned model, the controlling function 155 causes the display 140 to display the information indicating whether a sudden change in the clinical state is present or absent.

Alternatively, for example, the learned model may output a probability of the occurrence of a sudden change in the clinical state in the same manner as in the embodiments described above, so that the controlling function 155 judges whether a sudden change in the clinical state is present or absent according to the level of the probability output from the learned model and causes the display 140 to display information indicating whether a sudden change in the clinical state is present or absent on the basis of the judgment result.

In that situation, for example, either the learned model or the controlling function 155 determines that a sudden change in the clinical state is absent when the derived probability is lower than a predetermined threshold value (e.g., 10%) and determines that a sudden change in the clinical state is present when the derived probability is equal to or higher than the threshold value.

In another example, the information about the occurrence of a sudden change in the clinical state may be a score indicating a degree of certainty for the occurrence of a sudden change in the clinical state.

In that situation, for example, after deriving probability of the occurrence of a sudden change in the clinical state on the basis of the medical data input thereto, the learned model calculates and outputs a score indicating the level of the derived probability. After that, the controlling function 155 causes the display 140 to display information indicating the score output from the learned model.

In yet another example, the learned model may output a probability of the occurrence of a sudden change in the clinical state in the same manner as in the embodiments described above, so that the controlling function 755 calculates a score indicating the level of the probability output from the learned model and causes the display 140 to display information indicating the calculated score.

Alternatively, for example, instead of calculating the score from the probability, sudden changes in the clinical state may be learned as scores to begin with. In that situation, by performing a machine learning process by using learning-purpose data including the score, the learning function 154 may generate a learned model configured to output a score, as the information about the occurrence of a sudden change in the clinical state.

Alternatively, for example, instead of using the score, either the learned model or the controlling function 155 may indicate the level of the probability by using any one of a plurality of degrees such high, medium, and low.

Further, in the embodiments above, the example is explained in which, in the operation mode, the controlling function 155 causes the display 140 to display the information about the occurrence of a sudden change in the clinical state output from the learned model at a single point in time; however, possible embodiments are not limited to this example.

For instance, another arrangement is acceptable in which, in addition to the information output from the learned model at the single point in time, the controlling function 155 causes the display 140 to display information about the occurrence of a sudden change in the clinical state on the basis of pieces of information output from the learned model at a plurality of immediately-preceding points in time. In other words, the controlling function 155 may predict the occurrence of a sudden change in the clinical state by taking into account, not only the prediction result at the single point in time, but also the prediction results at the plurality of immediately-preceding points in time.

In that situation, for example, the controlling function 155 causes the display 140 to display a probability obtained by correcting the probability output from the learned model at the single point in time on the basis of probabilities output from the learned model at the plurality of immediately-preceding points in time.

For example, the controlling function 155 calculates an average value of the probability output from the learned model at the single point in time and the probabilities output from the learned model at the plurality of immediately-preceding points in time and further causes display 140 to display the average values. In that situation, the controlling function 155 may cause the display 140 to display only the average value based on the plurality of points in time or to display the probability at the single point in time simultaneously with the average value based on the plurality of points in time.

In yet another example, as explained above, when arranging the information indicating whether a sudden change in the clinical state present or absent to be displayed as the information about the occurrence of a sudden change in the clinical state, the controlling function 155 may judge whether a sudden change in the clinical state is present or absent by taking into account, not only the probability at a single point in time, but also probabilities at a plurality of immediately-preceding points in time.

In that situation, for example, on the basis of the probability at the single point in time and the probabilities at the plurality of immediately-preceding points in time, the controlling function 155 determines that a sudden change in the clinical state is present when two or more probabilities are consecutively equal to or higher than a predetermined threshold value (e.g., 10%) and determines that a sudden change in the clinical state is absent otherwise. In vet another example, the controlling function 155 may determine that a sudden change in the clinical state is present when two or more probabilities consecutively increase and determine that a sudden change in the clinical state is absent otherwise.

In this situation, for example, when displaying the list view arranged with the pieces of information obtained by simplifying the information contained in the screen illustrated in FIG. 8 with respect to the plurality of patients as explained above, the controlling function 155 may display the information of one or more patients determined to have a sudden change in the clinical state so as to be highlighted in the list.

Further, in e embodiments above, the example is explained in which the information about the medical data input to the learned model is either the statistical feature values or the relative values of the statistical feature values; however possible embodiments are not limited to this example. For instance, the information about the medical data input to the learned model may be the medical data itself. In other words, in that situation, the learned model further has the function of calculating the statistical feature values from the medical data input thereto. Further, in that situation, by inputting the medical data to the learned model, the controlling function 155 causes the learned model to output a probability of the occurrence of a sudden change in the clinical state.

Further, in the embodiments above, the example is explained in which the single apparatus, namely the medical information processing apparatus 100, performs the processes in both the learning mode and the operation mode; however, possible embodiments are not limited to this example. For instance, the processes in the learning mode for generating the learned model may be performed by another apparatus (hereinafter, “model generating apparatus”) different from the medical information processing apparatus 100. For example, the model generating apparatus is configured to obtain, from time to time, and accumulate the medical data generated by the electronic medical record system 500, the radiation department system 400, and the specimen testing system 500 and to generate or update the learned model regularly. In that situation, the model generating apparatus is configured to store therein the sudden change information used for generating the learned model, so that the medical information processing apparatus 100 arranges information indicating whether a sudden change in the clinical state actually occurred or not to be fed back to the model generating apparatus. Further, with predetermined timing or at a time when an instruction is received from the operator indicating that the model should be obtained, the medical information processing apparatus 100 obtains the learned model from the model generating apparatus and stores the obtained learned model into the storage 120.

Further, in the embodiments above, the example is explained in which the predetermined state change predicted by the medical information processing apparatus 100 is a sudden change in the clinical state; however, possible embodiments are not limited to this example. For instance, by using the same method as explained in any of the embodiments, the medical information processing apparatus 100 is also capable of predicting other state changes such as occurrence of a side effect of treatment or deterioration of the clinical state (e.g., a postoperative infection) caused by a factor that is not directly related to a treated disease.

In the embodiments above, the example is explained in which the first obtaining unit, the second obtaining unit, the generating unit, the learning unit, and the controlling unit of the present disclosure are realized by the first obtaining function 151, the second obtaining function 152, the generating function 153, the learning function 154, and the controlling function 155 of the processing circuitry 150, respectively; however, possible embodiments are not limited to this example. For instance, instead of realizing the first obtaining unit, the second obtaining unit, the generating unit, the learning unit, and the controlling unit of the present disclosure with the first obtaining function 151, the second obtaining function 152, the generating function 153, the learning function 154, and the controlling function 155 described in the embodiments, it is also acceptable to realize the same functions by using only hardware or by using a combination of hardware and software.

The term “processor” used in the above explanations denotes, for example, a Central Processing Unit (CPU), a Graphics Processing Unit (GPU), or a circuit such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). The one or more processors realize the functions by reading and executing the programs saved in the storage 120. In this situation, instead of saving the programs in the storage 120, it is also acceptable to directly incorporate the programs in the circuits of the processors. In that situation, the processors realize the functions by reading and executing the programs incorporated in the circuits thereof. Further, the processors in the present embodiments do not each necessarily have to be structured as a single circuit. It is also acceptable to structure one processor by combining together a plurality of independent circuits so as to realize the functions thereof.

In this situation, the programs executed by the processors are provided as being incorporated, in advance, into a Read-Only Memory (ROM) or a storage, for example. Alternatively, the programs may be provided as being recorded on a computer-readable storage medium such as a Compact Disk Read-Only Memory (CD-ROM), a flexible disk (FD), a Compact Disk Recordable (CD-R), a Digital Versatile Disk (DVD), or the like, in a file in such a format that is either installable or executable for the devices. Further, the programs may be stored in a computer connected to a network such as the Internet, so as to be provided or distributed as being downloaded via the network. For example, the programs are structured with modules including the functional units described above. In the actual hardware, as a result of a CPU reading and executing the programs from a storage medium such as a ROM, the modules are loaded into a main storage device so as to be generated in the main storage device.

According to at least one aspect of the embodiments described above, it is possible to more accurately predict the advance indication of a sudden change in the clinical state.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing apparatus comprising:
processing circuitry configured to
obtain, from among medical data related to subjects, medical data in a specific time period at or earlier than a previous point in time that is earlier by a predetermined length of time than a time of occurrence of a predetermined state change in the subjects; and
generate, on a basis of information about the medical data in the specific time period, learning-purpose data used for generating a learned model configured to output information about the predetermined state change in a subject being subject to a prediction that may occur at a time later by the predetermined length of time than a time at which a medical service is provided for the subject.

2. The medical information processing apparatus according to claim 1, wherein the processing circuitry is further configured to
obtain sudden change information indicating whether the predetermined state change is present or absent in the subjects, and
generate, as the learning-purpose data, data including the information about the medical data in the specific time period and the sudden change information.

3. The medical information processing apparatus according to claim 1, wherein the specific time period is a certain time period included in a day that is earlier by a predetermined number of days than a day of the occurrence of the predetermined state change.

4. The medical information processing apparatus according to claim 1, wherein the specific time period is represented by a plurality of mutually-different time periods on or earlier than a previous date that is earlier by a predetermined number of days than a day of the occurrence of the predetermined state change.

5. The medical information processing apparatus according to claim 1, wherein the specific time period is a time period selected in accordance with the information about the medical data from among a plurality of mutually-different time periods on or earlier than a previous date that is earlier by a predetermined number of days than a day of the occurrence of the predetermined state change.

6. A medical information processing apparatus comprising:
processing circuitry configured to
obtain medical data at or earlier than a time at which a medical service is provided for a subject; and
exercise control by inputting information about the obtained medical data to a learned model and causing the learned model to output information about a predetermined state change in the subject that may occur at a time later by a predetermined length of time than a time at which the medical service is provided, the learned model being configured to receive an input of information about medical data at or earlier than a time at which a medical service is provided for a subject being subject to a prediction and configured to output information about the predetermined state change in the subject that may occur at a time later by the predetermined length of time than the time of the medical service.

7. The medical information processing apparatus according to claim 6, wherein the processing circuitry is further configured to generate the learned model by performing a machine learning process while using, as learning-purpose data, the information about the medical data at or earlier than the time at which the medical service is provided for the subject and information about the occurrence of the predetermined state change in the subject.

8. The medical information processing apparatus according to claim 7, wherein the processing circuitry is further configured to receive, from an operator, an operation to input information indicating whether or not the predetermined state change actually occurred in response to the information output from the learned model and further arranges a result that has been input to be fed back to the information about the occurrence of the predetermined state change.

9. A medical information processing apparatus comprising:
the processing circuitry configured to cause a display to display information about medical data at or earlier than a time at which a medical service is provided for a subject and information about a predetermined state change in the subject that may occur at a time that is later by a predetermined length of time than the time of the medical service.

10. The medical information processing apparatus according to claim 9, wherein the processing circuitry is further configured to cause the display to display information about medical data serving as basis of the information about the occurrence of the predetermined state change.

11. The medical information processing apparatus according to claim 10, wherein the processing circuitry is further configured to cause the display to display information indicating a chronological trajectory of the information about the medical data serving as the basis of the information about the occurrence of the predetermined state change.

12. The medical information processing apparatus according to claim 1, wherein the information about the occurrence of the predetermined state change is a probability of the occurrence of the predetermined state change; information indicating whether the predetermined state change is present or absent; or a score indicating a degree of certainty for the occurrence of the predetermined state change.

13. The medical information processing apparatus according to claim 6, wherein the information about the occurrence of the predetermined state change is a probability of the occurrence of the predetermined state change; information indicating whether the predetermined state change is present or absent; or a score indicating a degree of certainty for the occurrence of the predetermined state change.

14. The medical information processing apparatus according to claim 9, wherein the information about the occurrence of the predetermined state change is a probability of the occurrence of the predetermined state change; information indicating whether the predetermined state change is present or absent; or a score indicating a degree of certainty for the occurrence of the predetermined state change.

15. The medical information processing apparatus according to claim 1, wherein the information about the medical data is a statistical feature value calculated from medical data in a specific time period at or earlier than the time at which the medical service is provided for the subject.

16. The medical information processing apparatus according to claim 6, wherein the information about the medical data is a statistical feature value calculated from medical data in a specific time period at or earlier than the time at which the medical service is provided subject.

17. The medical information processing apparatus according to claim 9, wherein the information about the medical data is a statistical feature value calculated from medical data in a specific time period at or earlier than the time at which the medical service is provided for the subject.

18. The medical information processing apparatus according to claim 1, wherein the information about the medical data is a relative value between a statistical feature value calculated from medical data in a specific time period at or earlier than the time at which the medical service is provided for the subject and a statistical feature value calculated from medical data in a reference time period determined for each of subjects including the subject.

19. The medical information processing apparatus according to claim 6, wherein the information about the medical data is a relative value between a statistical feature value calculated from medical data in a specific time period at or earlier than the time at which the medical service is provided for the subject and a statistical feature value calculated from medical data in a reference time period determined for each of subjects including the subject.

20. The medical information processing apparatus according to claim 9, wherein the information about the medical data is a relative value between a statistical feature value calculated from medical data in a specific time period at or earlier than the time at which the medical service is provided for the subject and a statistical feature value calculated from medical data in a reference time period determined for each of subjects including the subject.
